(19) United States
(12) Patent Application Publication
(10) Pub. No.: US 2013/0241055 A1
(43) Pub. Date: Sep. 19, 2013

(54) Multi-Chip Packages and Methods of Manufacturing the Same

(71) Applicant: SAMSUNG ELECTRONICS CO., LTD., Suwon-si (KR)

(72) Inventors: Jin-Young Jung, Hwaseong-si (KR); Do-ll Kong, Hwaseong-si (KR); Hai-lck Kim, Daegu (KR)

(73) Assignee: Samsung Electronics Co., Ltd., Suwon-si (KR)

(21) Appl. No.: 13/783,484

(22) Filed: Mar. 4, 2013

(30) Foreign Application Priority Data
Mar. 14, 2012 (KR) 10-2012-0025911

(51) Int. Cl. H01L 23/00; H01L 21/82; H01L 23/498

(52) U.S. Cl. CPC H01L 23/562; H01L 23/49811; H01L 21/82
USPC 257/737; 438/107

(57) ABSTRACT

Multi-chip packages are provided having a first semiconductor chip arranged on a package substrate. The first semiconductor chip includes a first bonding pad connected to the package substrate. A second semiconductor chip is arranged on the first semiconductor chip. The second semiconductor chip has an overhang that protrudes from a side surface of the first semiconductor chip, and a second bonding pad arranged on the overhang. A third semiconductor chip is arranged on the second semiconductor chip to expose the overhang. The third semiconductor chip has a third bonding pad. A first conductive wire may be connected between the second bonding pad and the third bonding pad. A second conductive wire may be connected between the third bonding pad and the package substrate.

### REFERENCE TO PRIORITY APPLICATION

This application claims priority to Korean Patent Application No. 10-2012-0025911, filed on Mar. 14, 2012, the contents of which are hereby incorporated herein by reference as if set forth in its entirety.

### FIELD

The present inventive concept relates generally to semiconductor packages, and, more particularly, to multi-chip semiconductor packages including sequentially stacked semiconductor chips and related methods of manufacturing.

### BACKGROUND

Generally, multiple semiconductor fabrication processes may be performed on a semiconductor substrate to form a plurality of semiconductor chips. In order to mount the semiconductor chips on a printed circuit board (PCB), a packaging process may be performed on the semiconductor chips to form semiconductor packages.

In order to increase storage capacity of the semiconductor package, a multi-chip package including sequentially stacked semiconductor chips has been developed. The stacked semiconductor chips may be electrically connected via conductive wires.

The multi-chip package may generally include a package substrate; a first semiconductor chip arranged on an upper surface of the package substrate; a second semiconductor chip arranged on an upper surface of the first semiconductor chip; and a third semiconductor chip arranged on an upper surface of the second semiconductor chip. The second semiconductor chip may have an overhang that protrudes from a side surface of the first semiconductor chip. First bonding pads may be arranged on an upper edge surface of the first semiconductor chip. Second bonding pads may be arranged on an upper surface of the overhang of the second semiconductor chip. Third bonding pads may be arranged on an upper edge surface of the third semiconductor chip.

First conductive wires may be electrically connected between the first bonding pads and the package substrate. Second conductive wires may be electrically connected between the first bonding pads and the second bonding pads. Third conductive wires may be electrically connected between the second bonding pads and the third bonding pads. Thus, the third semiconductor chip may be electrically connected to the package substrate via the second semiconductor chip and the first semiconductor chip. Therefore, the second conductive wires and the third conductive wires may be connected to the overhang of the second semiconductor chip. Accordingly, at least two wire bonding processes may be performed on the overhang.

However, there may not be any structure to support the overhang. The at least two wire bonding processes may apply ample stress to the overhang, which may generate cracks in the overhang and the second semiconductor chip.

In order to reduce the likelihood of, or possibly prevent, cracks from being generated, the second semiconductor chip may have a stronger overhang than the overhang of the first semiconductor chip. In order to reinforce the strength of the second semiconductor chip, the second semiconductor chip including the overhang may have a thickness greater than that of the first semiconductor chip and the third semiconductor chip, which may result in increasing a thickness of the multi-chip package.

### SUMMARY

Some embodiments of the present inventive concept provide multi-chip packages having a thin thickness capable of suppressing stresses of a wire bonding process and related methods of manufacturing multi-chip packages.

Further embodiments of the present inventive concept provide multi-chip packages including a package substrate, a first semiconductor chip, a second semiconductor chip, a third semiconductor chip, a first conductive wire and a second conductive wire. The first semiconductor chip is arranged on an upper surface of the package substrate. The first semiconductor chip has a first bonding pad electrically connected with the package substrate. The second semiconductor chip is arranged on an upper surface of the first semiconductor chip. The second semiconductor chip has an overhang that protrudes from a side surface of the first semiconductor chip, and a second bonding pad arranged on the overhang. The third semiconductor chip is arranged on an upper surface of the second semiconductor chip to expose the overhang. The third semiconductor chip has a third bonding pad. The first conductive wire is electrically connected between the second bonding pad and the third bonding pad. The second conductive wire is electrically connected between the third bonding pad and the package substrate.

In still further embodiments, the multi-chip package may further include a fourth semiconductor chip, a third conductive wire and a fourth conductive wire. The fourth semiconductor chip may be between the package substrate and the first semiconductor chip. The fourth semiconductor chip may have a fourth bonding pad. The third conductive wire may be electrically connected between the first bonding pad and the fourth bonding pad. The fourth conductive wire may be electrically connected between the fourth bonding pad and the package substrate.

In some embodiments, the multi-chip package may further include a fifth semiconductor chip and a fifth conductive wire. The fifth semiconductor chip may be arranged on an upper surface of the third semiconductor chip. The fifth semiconductor chip may have a fifth bonding pad electrically connected to the second conductive wire. The fifth conductive wire may be electrically connected between the fifth bonding pad and the package substrate.

In further embodiments, the multi-chip package may further include a fifth semiconductor chip and a fifth conductive wire. The fifth semiconductor chip may be arranged on a first surface of the third semiconductor chip. The fifth semiconductor chip may have a fifth bonding pad. The fifth conductive wire may be electrically connected between the third bonding pad and the fifth bonding pad.

In still further embodiments, the first bonding pad may be arranged on a first edge portion of the upper surface of the first semiconductor chip adjacent to the overhang. The second semiconductor chip may be arranged on the upper surface of the first semiconductor chip to cover the first bonding pad.

In some embodiments, the first bonding pad may be arranged on a second edge portion of the first surface of the first semiconductor chip opposite to the overhang. The second semiconductor chip may be arranged on the first surface of the first semiconductor chip to expose the first bonding pad.

In further embodiments, the multi-chip package may further include a first pad bump, a second pad bump and a third pad bump. The first pad bump may be formed on the third bonding pad. The first pad bump may be electrically connected to an upper end of the first conductive wire. The second pad bump may be formed on the first pad bump. The second pad bump may be electrically connected to the second conductive wire. The third pad bump may be formed on the second bonding pad. The third pad bump may be electrically connected to a lower end of the first conductive wire.

In still further embodiments, a thickness of the second semiconductor chip may be no greater than a thickness of the third semiconductor chip.

In some embodiments, the multi-chip package may further include a molding member and external terminals. The molding member may be on the upper surface of the package substrate to cover the first semiconductor chip, the second semiconductor chip and the third semiconductor chip. The external terminals may be mounted on a lower surface of the package substrate.

Further embodiments of the present inventive concept provide methods of manufacturing multi-chip packages including arranging a first semiconductor chip on a first surface of a package substrate. A second semiconductor chip is arranged on a first surface of the first semiconductor chip. The second semiconductor chip has an overhang that protrudes from a side surface of the first semiconductor chip, and a second bonding pad arranged on the overhang. The third semiconductor chip is arranged on an upper surface of the second semiconductor chip to expose the overhang. The third semiconductor chip has a third bonding pad. A first conductive wire is electrically connected between the second bonding pad and the third bonding pad. A second conductive wire is electrically connected between the third bonding pad and the package substrate.

In still further embodiments, the method may further include providing a fourth semiconductor chip having a fourth bonding pad between the package substrate and the first semiconductor chip, electrically connecting the first bonding pad with the fourth bonding pad using a third conductive wire, and electrically connecting the fourth bonding pad with the package substrate using a fourth conductive wire.

In further embodiments, the method may further include arranging a fifth semiconductor chip having a fifth bonding pad on an upper surface of the third semiconductor chip, and electrically connecting the fifth bonding pad with the third bonding pad using a fifth conductive wire.

In still further embodiments, the method may further include arranging a fifth semiconductor chip having a fifth bonding pad on an upper surface of the third semiconductor chip, electrically connecting the second conductive wire to the fifth bonding pad, and electrically connecting the fifth bonding pad with the package substrate using a fifth conductive wire.

### BRIEF DESCRIPTION OF THE DRAWINGS

Example embodiments will be more clearly understood from the following detailed description taken in conjunction with the accompanying drawings. FIGS. 1 to 24 represent non-limiting, example embodiments as described herein.

FIG. 1 is a cross-section illustrating a multi-chip package in accordance with some embodiments of the present inventive concept.

FIG. 2 is a perspective view illustrating a wire bonding structure between semiconductor chips of the multi-chip package of FIG. 1 in accordance with some embodiments of the present inventive concept.

FIG. 3 is an enlarged cross-section a portion III of FIG. 1 in accordance with some embodiments of the present inventive concept.

FIGS. 4 to 9 are cross-sections illustrating processing step in the fabrication of multi-chip packages illustrated in FIG. 1 in accordance with some embodiments of the present inventive concept.

FIG. 10 is an enlarged cross-section illustrating semiconductor chips of a multi-chip package in accordance with some embodiments of the present inventive concept.

FIG. 11 is an enlarged cross-section of a portion XI of FIG. 10 in accordance with some embodiments of the present inventive concept.

FIGS. 12 to 16 are cross-sections illustrating processing steps in the fabrication of multi-chip packages of FIG. 10 in accordance with some embodiments of the present inventive concept.

FIG. 17 is an enlarged cross-section illustrating a multi-chip package in accordance with some embodiments of the present inventive concept.

FIGS. 18 and 19 are cross-sections illustrating processing steps in the fabrication of the multi-chip package in FIG. 17 in accordance with some embodiments of the present inventive concept.

FIG. 20 is a cross-section illustrating a multi-chip package in accordance with some embodiments of the present inventive concept.

FIG. 21 is a cross-section illustrating a multi-chip package in accordance with in accordance with some embodiments of the present inventive concept.

FIG. 22 is a cross-section illustrating a multi-chip package in accordance with some embodiments of the present inventive concept.

FIG. 23 is a cross-section illustrating a multi-chip package in accordance with some embodiments of the present inventive concept.

FIG. 24 is a cross-section illustrating a multi-chip package in accordance with some embodiments of the present inventive concept.

### DETAILED DESCRIPTION OF THE EMBODIMENTS

Various example embodiments will be described more fully hereinafter with reference to the accompanying drawings, in which some example embodiments are shown. The present inventive concept may, however, be embodied in many different forms and should not be construed as limited to the example embodiments set forth herein. Rather, these example embodiments are provided so that this disclosure will be thorough and complete, and will fully convey the scope of the present inventive concept to those skilled in the art. In the drawings, the sizes and relative sizes of layers and regions may be exaggerated for clarity.

It will be understood that when an element or layer is referred to as being “on,” “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numerals refer to like elements throughout. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present inventive concept.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular example embodiments only and is not intended to be limiting of the present inventive concept. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Example embodiments are described herein with reference to cross-sectional illustrations that are schematic illustrations of idealized example embodiments (and intermediate structures). As such, variations from the shapes of the illustrations as a result, for example, of manufacturing techniques and/or tolerances, are to be expected. Thus, example embodiments should not be construed as limited to the particular shapes of regions illustrated herein but are to include deviations in shapes that result, for example, from manufacturing. For example, an implanted region illustrated as a rectangle will, typically, have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of the present inventive concept.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

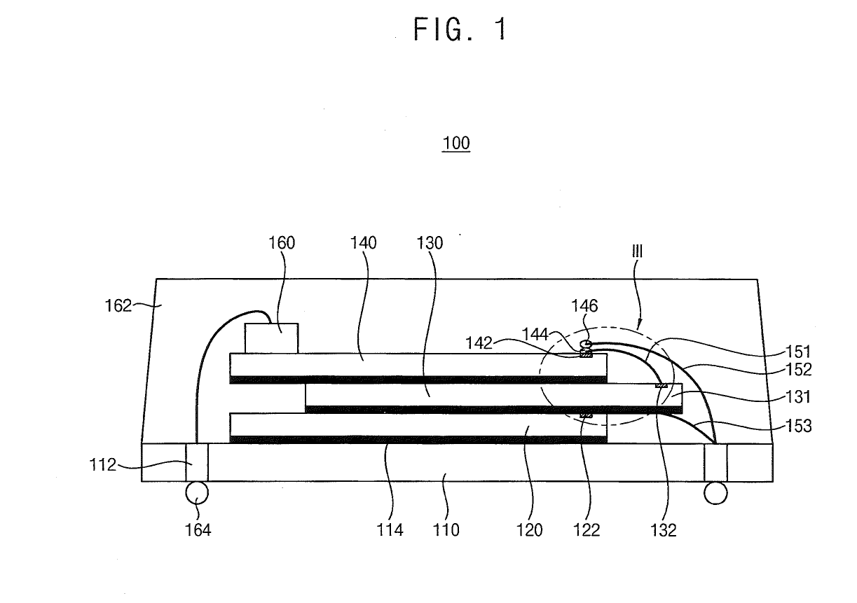

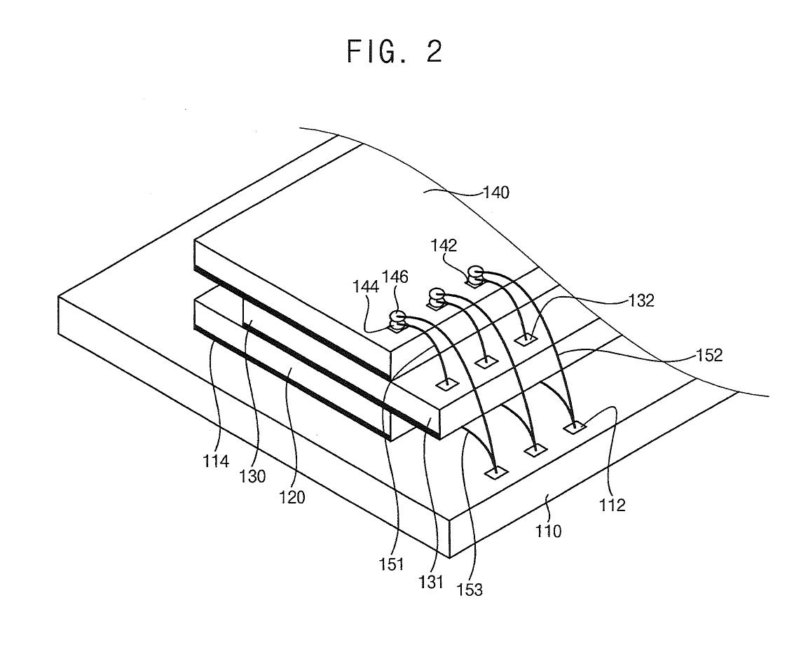

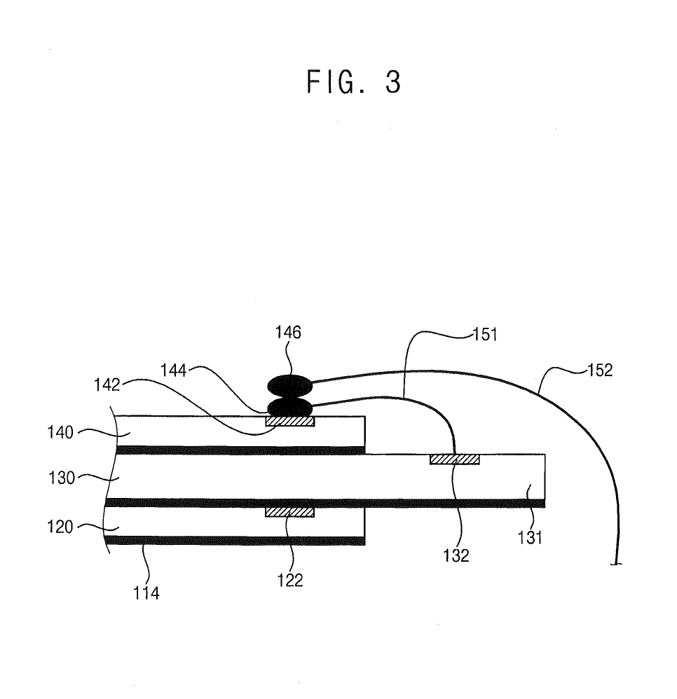

FIG. 1 is a cross-section illustrating a multi-chip package in accordance with example embodiments; FIG. 2 is a perspective view illustrating a wire bonding structure between semiconductor chips of the multi-chip package in FIG. 1; and FIG. 3 is an enlarged cross-section of portion III of FIG. 1. Referring first to FIG. 1, a multi-chip package 100 in accordance with some embodiments may include a package substrate 110, a first semiconductor chip 120, a second semiconductor chip 130, a third semiconductor chip 140, a first conductive wire 151, a second conductive wire 152, a third conductive wire 153, a control chip 160, a molding member 162 and external terminals 164.

The package substrate 110 may be, for example, an insulating substrate, and a circuit pattern 112 may be built in the insulating substrate 110. The circuit pattern 112 may have a first end exposed through a first surface of the insulating substrate 110, and a second end exposed through as second surface, opposite the first surface, of the insulating substrate 110.

The first semiconductor chip 120, the second semiconductor chip 130 and the third semiconductor chip 140 may be stacked on the first surface of the package substrate 110. In some embodiments, the first semiconductor chip 120 may be arranged on the first surface of the package substrate 110. The second semiconductor chip 130 may be arranged on a surface of the first semiconductor chip 120. The third semiconductor chip 140 may be arranged on a surface of the second semiconductor chip 130. The first to third semiconductor chips 120, 130 and 140 may be attached using an adhesive 114.

In some embodiments, the second semiconductor chip 130 may have an overhang 131 that protrudes horizontally from a side surface of the first semiconductor chip 120. The overhang 131 may be cantilevered over a surface of the substrate 110 such that an empty space may be formed under the overhang 131. The overhang 131 may have a weak structure because a support for supporting the overhang 131 may not exist.

In some embodiments, the third semiconductor chip 140 may be arranged on a surface of the second semiconductor chip 130 to expose a surface of the overhang 131. The third semiconductor chip 140 may have a side surface substantially coplanar with a side surface of the first semiconductor chip 120.

The first semiconductor chip 120 may have a first bonding pad 122. The second semiconductor chip 130 may have a second bonding pad 132. The third semiconductor chip 140 may have a third bonding pad 142. The first bonding pad 122 may be arranged on an edge portion of a surface of the first semiconductor chip 120. The second bonding pad 132 may be arranged on an edge portion of a surface of the second semiconductor chip 130. The third bonding pad 142 may be arranged on an edge portion of a surface of the third semiconductor chip 140. Thus, in some embodiments, the first bonding pad 122 may be covered with a lower surface of the second semiconductor chip 130. In contrast, the second bonding pad 132 may be arranged on a surface of the overhang 131, so that the second bonding pad 132 may be exposed.

Referring now to FIGS. 1 and 2, the first bonding pad 122 of the first semiconductor chip 120 may be electrically connected to the circuit pattern 112 of the package substrate 110 via the third conductive wire 153. The second bonding pad 132 of the second semiconductor chip 130 may be electrically connected to the third bonding pad 142 of the third semiconductor chip 140 via the first conductive wire 151. The third bonding pad 142 of the third semiconductor chip 140 may be electrically connected to the circuit pattern 112 of the package substrate 110 via the second conductive wire 152.

In some embodiments, only one end of the first conductive wire 151 may be connected to the second bonding pad 132 on the overhang 131 of the second semiconductor chip 130 without the support. In contrast, an end of the first conductive wire 151 and an end of the second conductive wire 152 may be connected to the third bonding pad 142 of the third semiconductor chip 140 supported by the second semiconductor chip 130. In other words, the second semiconductor chip 130 having the overhang 131 may not be directly connected to the package substrate 110. The second semiconductor chip 130 may be indirectly connected to the package substrate 110 through the third semiconductor chip 140 over the second semiconductor chip 130.

Accordingly, only one wire bonding process may be performed on the overhang 131 of the second semiconductor chip 130 having the weak structure. Thus, stress applied to the overhang 131 caused by the wire bonding process may be greatly reduced. As a result, the likelihood that cracks will form in the second semiconductor chip 130 having the overhang 131 may be reduced. Furthermore, the second semiconductor chip 130 having the overhang 131 may not need to be stronger than the first semiconductor chip 120 and the third semiconductor chip 140. Thus, the second semiconductor chip 130 may have a thickness substantially equal to or less than that of the first semiconductor chip 120 and the third semiconductor chip 140. The thickness of the second semiconductor chip 130 may be no greater than a thickness of the first semiconductor chip 120 and the third semiconductor chip 140. As a result, the presence of the second semiconductor chip 130 having the overhang 131 may not increase a total thickness of the multi-chip package 100.

Referring now to FIG. 3, an enlarged cross-section of a portion III of FIG. 1 will be used to discuss wire bonding structures between the second semiconductor chip 130 and the third semiconductor chip 140. As illustrated in FIG. 3, a first pad bump 144 may be formed on the third bonding pad 142. The first conductive wire 151 may be electrically connected between the first pad bump 144 and the second bonding pad 132. Thus, the first conductive wire 151 may a first end connected to the first pad bump 144, and a second end connected to the second bonding pad 132.

A second pad bump 146 may be formed on the first pad bump 144. The second conductive wire 152 may be electrically connected between the second pad bump 146 and the circuit pattern 112 of the package substrate 110. Thus, the second conductive wire 152 may have a first end connected to the second pad bump 146, and a second end connected to the circuit pattern 112 of the package substrate 110.

Referring again to FIG. 1, the control chip 160 may be arranged on the upper surface of the third semiconductor chip 140. The control chip 160 may be electrically connected with the circuit pattern 112 of the package substrate 110 via a conductive wire 161.

The molding member 162 may be formed on the upper surface of the package substrate 110 to cover the first semiconductor chip 120, the second semiconductor chip 130 and the third semiconductor chip 140. The molding member 162 may protect the first to third semiconductor chips 120, 130 and 140 and the first to third conductive wires 151, 152 and 153 from external environments. In some embodiments, the molding member 162 may include an epoxy molding compound (EMC).

The external terminals 164 may be mounted on the lower end of the circuit pattern 112 exposed through the lower surface of the package substrate 110. In some embodiments, the external terminals 164 may include solder bumps.

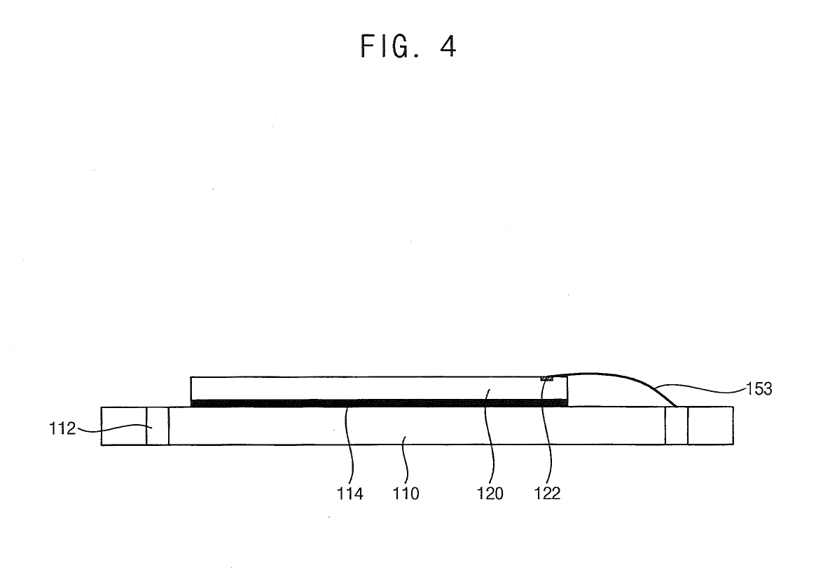

Referring now to FIGS. 4 to 9, cross-sections illustrating processing steps in the fabrication of multi-chip packages illustrated in FIG. 1 will be discussed. As illustrated in FIG. 4, the first semiconductor chip 120 may be attached to the upper surface of the package substrate 110 using the adhesive 114. The first bonding pad 122 of the first semiconductor chip 120 may be electrically connected with the circuit pattern 112 of the package substrate 110 using the third conductive wire 153. In some embodiments, the first bonding pad 122 may be positioned on the right edge portion of the upper surface of the first semiconductor chip 120.

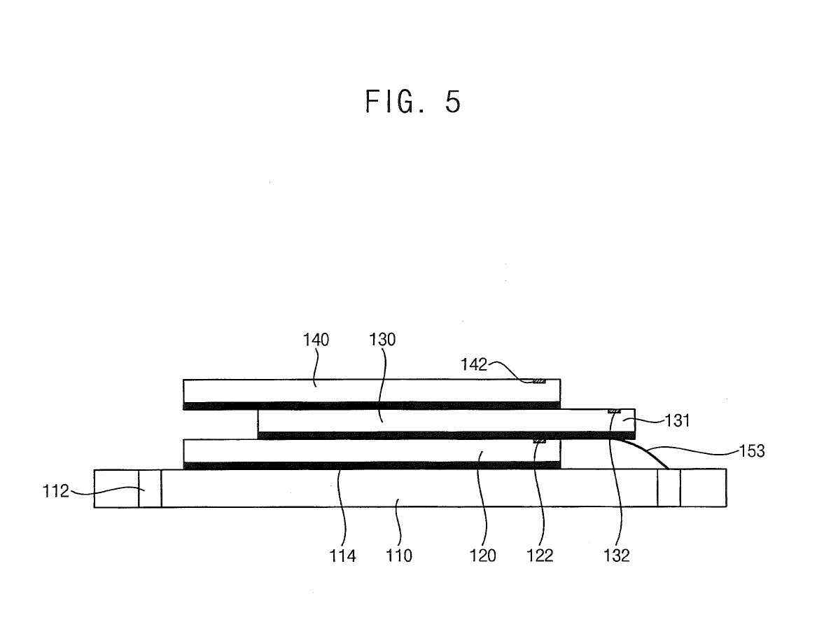

Referring now to FIG. 5, the second semiconductor chip 130 may be attached to the upper surface of the first semiconductor chip 120 using the adhesive 114. In some embodiments, the second semiconductor chip 130 may cover the first bonding pad 122 of the first semiconductor chip 120. Furthermore, the second semiconductor chip 130 may have the overhang 131 that protrudes from a first side surface of the first semiconductor chip 120. The second bonding pad 132 of the second semiconductor chip 130 may be on a surface, for example, an upper surface of the overhang 131.

The third semiconductor chip 140 may be attached to the surface of the second semiconductor chip 130 using the adhesive 114. In some embodiments, the overhang 131 may not be covered with the third semiconductor chip 140. In other words, in some embodiments, the overhang 131 may be exposed in an upward direction. Thus, the second bonding pad 132 may also be exposed by the third semiconductor chip 140. The third bonding pad 142 may be arranged on the right edge portion of the upper surface of the third semiconductor chip 140.

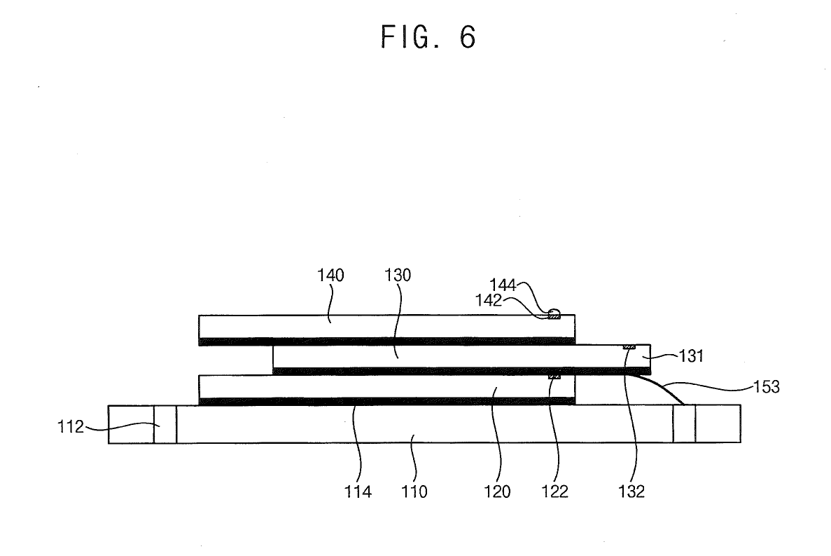

Referring now to FIG. 6, the first pad bump 144 may be formed on the third bonding pad 142. In some embodiments, the first pad bump 144 may be formed by, for example, applying an ultrasonic wave to a lower end of a metal wire drawn through a capillary.

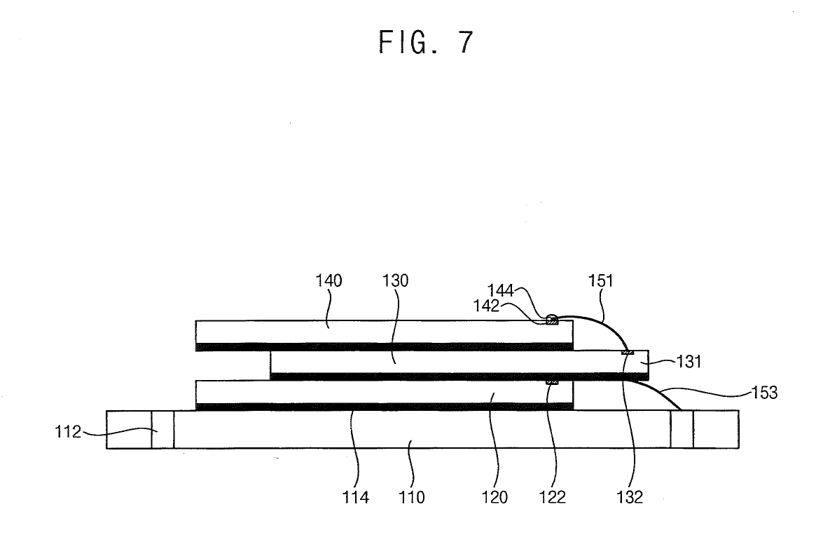

Referring now to FIG. 7, the metal wire drawn through the capillary may be extended from the first pad bump 144 to the second bonding pad 132 to form the first conductive wire 151 for electrically connecting the first pad bump 144 with the second bonding pad 132. Thus, the first conductive wire 151 may have the upper end connected to the first pad bump 144, and the lower end connected to the second bonding pad 132.

In some embodiments, only one wire bonding process for connecting the lower end of the first conductive wire 151 to the second bonding pad 132 may be performed on the overhang 131 having a relatively weak structure. Thus, stresses applied to the weak overhang 131 may be reduced. As a result, generations of cracks in the second semiconductor chip 130 may be suppressed. Therefore, the second semiconductor chip 130 may not require strength greater than that of the first semiconductor chip 120, so that the second semiconductor chip 120 may have a thickness of no more than a thickness of the first semiconductor chip 120.

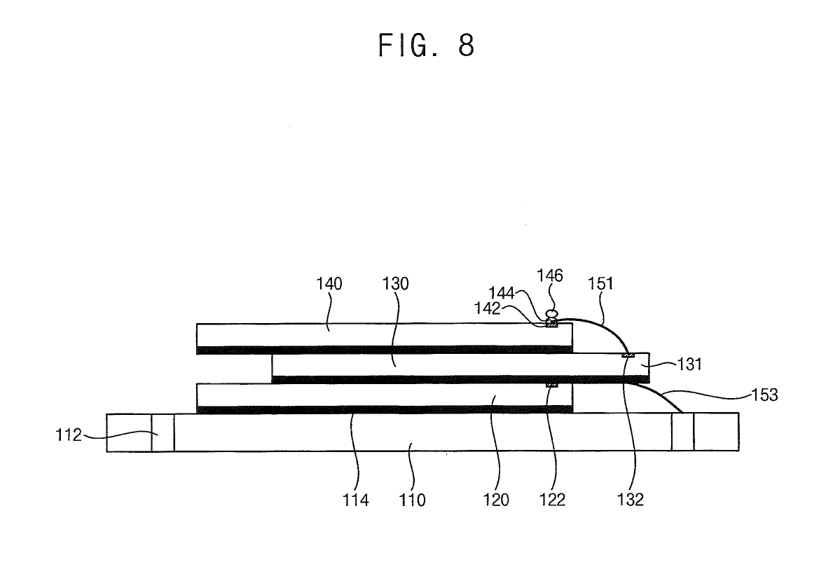

Referring now to FIG. 8, the second pad bump 146 may be formed on the first pad bump 144. In some embodiments, the second pad bump 146 may be formed by a process substantially the same as that for forming the first pad bump 144.

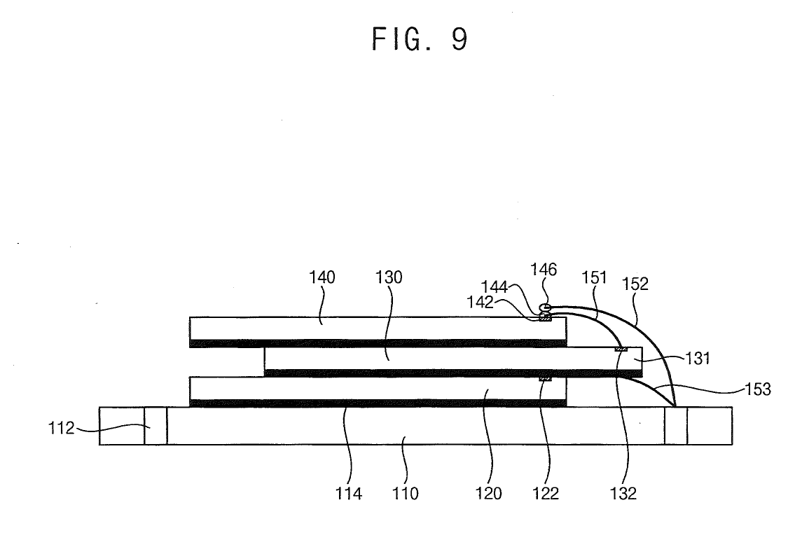

Referring to FIG. 9, the metal wire drawn through the capillary may be extended from the second pad bump 146 to the circuit pattern 112 of the package substrate 110 to form the second conductive wire 152 for electrically connecting the second pad bump 146 to the circuit pattern 112 of the package substrate 110. Thus, the second conductive wire 152 may have a first end connected to the second pad bump 146, and a second end connected to the circuit pattern 112 of the package substrate 110.

In some embodiments, two wire bonding processes for connecting the first conductive wire 151 and the second conductive wire 152 to the third bonding pad 142, respectively, may be performed on the third bonding pad 142 of the third semiconductor chip 140 firmly supported by the second semiconductor chip 130. Although the two wire bonding processes may be performed on the third semiconductor chip 140, cracks may not be generated in the third semiconductor chip 140 because the third semiconductor chip 140 may be firmly supported by the second semiconductor chip 130.

The control chip 160 may be attached to the upper surface of the third semiconductor chip 140. The control chip 160 and the package substrate 110 may be electrically connected with each other using the conductive wire 161. The molding member 162 may be formed on the upper surface of the package substrate 110 to cover the first to third semiconductor chips 120, 130 and 140, and the first to third conductive wires 151, 152 and 153. The external terminals 164 may be mounted on the circuit pattern 112 exposed through the lower surface of the package substrate 110 to complete embodiments of the multi-chip package 100 illustrated in FIG. 1.

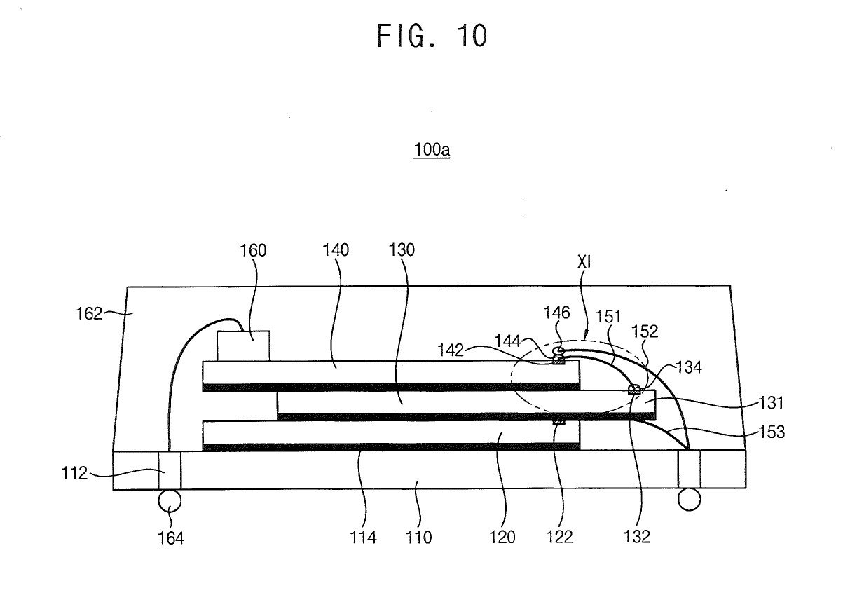

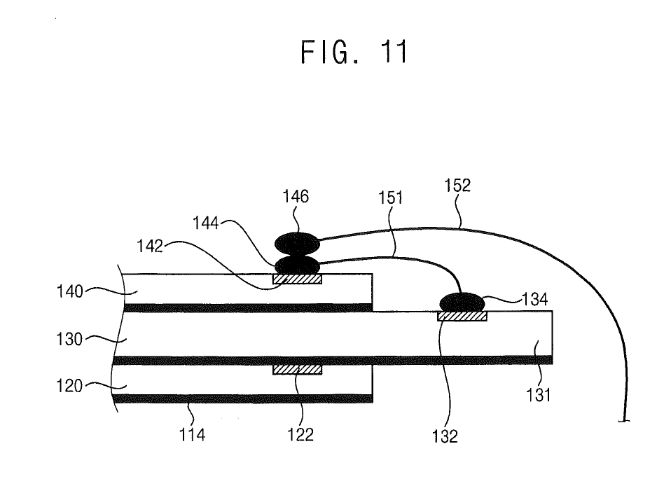

FIG. 10 is an enlarged cross-section illustrating semiconductor chips of a multi-chip package in accordance with some embodiments of the present inventive concept. FIG. 11 is an enlarged cross-section of a portion XI of FIG. 10. A multi-chip package 100a of embodiments illustrated in FIGS. 10 and 11 may include elements substantially the same as those of the multi-chip package 100 illustrated in FIG. 1, except embodiments illustrated in FIGS. 10 and 11 may include a third pad bump. It will be understood that the same reference numerals refer to the same elements throughout and, therefore, details of elements already discussed with respect to previous embodiments will not be discussed again in the interest of brevity.

Referring now to FIGS. 10 and 11, the multi-chip package 100a includes a third pad bump 134. The third pad bump 134 may be formed on the second bonding pad 132 of the second semiconductor chip 130. A first end of the first conductive wire 151 may be connected to the third pad bump 134. In some embodiments, a second end of the first conductive wire 151 may be connected to the third pad bump 134 having an area larger than that of the second bonding pad 132, so that the third pad bump 134 may improve electrical connection reliability between the first conductive wire 151 and the second bonding pad 132.

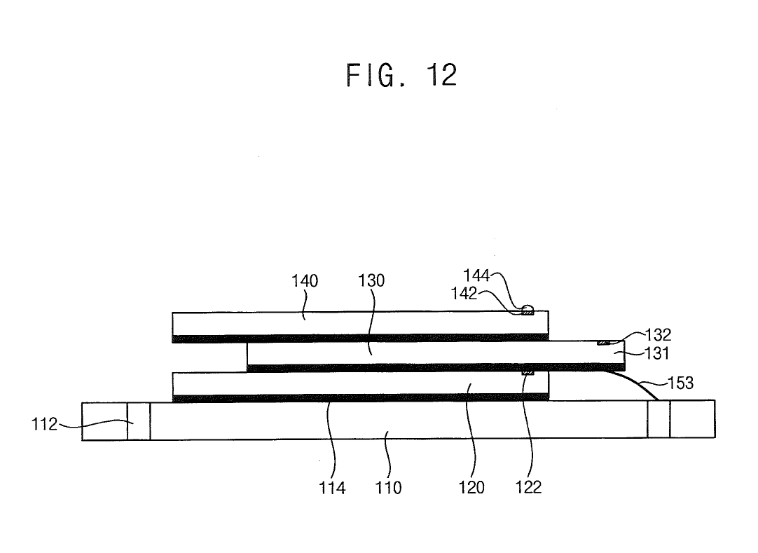

Referring now to FIGS. 12 to 16, cross-sections illustrating processing steps in the fabrication of multi-chip packages in accordance with embodiments illustrated in FIG. 10 will be discussed. Processes substantially the same as those illustrated with reference to FIGS. 4 and 5 may be performed to sequentially stack the first semiconductor chip 120, the second semiconductor chip 130 and the third semiconductor chip 140 on the package substrate 110. In some embodiments, the first semiconductor chip 120 may be electrically connected with the package substrate 110 via the third conductive wire 153.

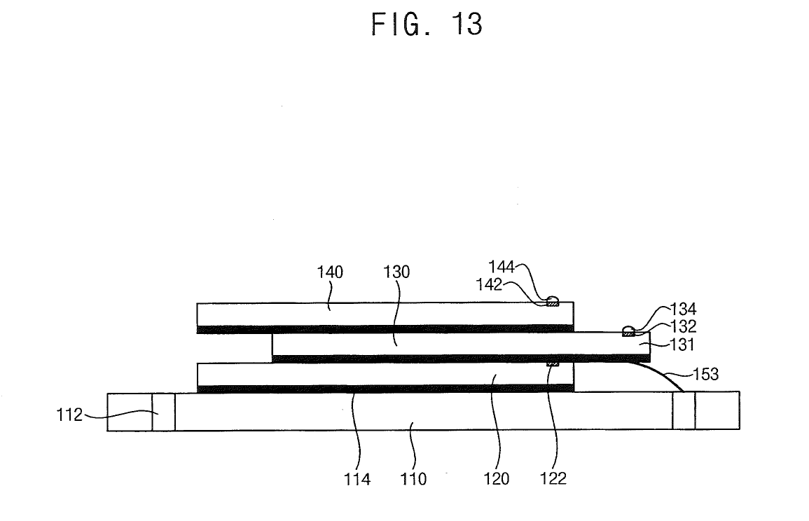

As illustrated in FIG. 12, the first pad bump 144 may be formed on the third bonding pad 142. As illustrated in FIG. 13, the third pad bump 134 may be formed on the second bonding pad 132. In some embodiments, the third pad bump 134 may be formed by a process substantially the same as a process for forming the first pad bump 144 and, thus, a detailed description of this process will be omitted.

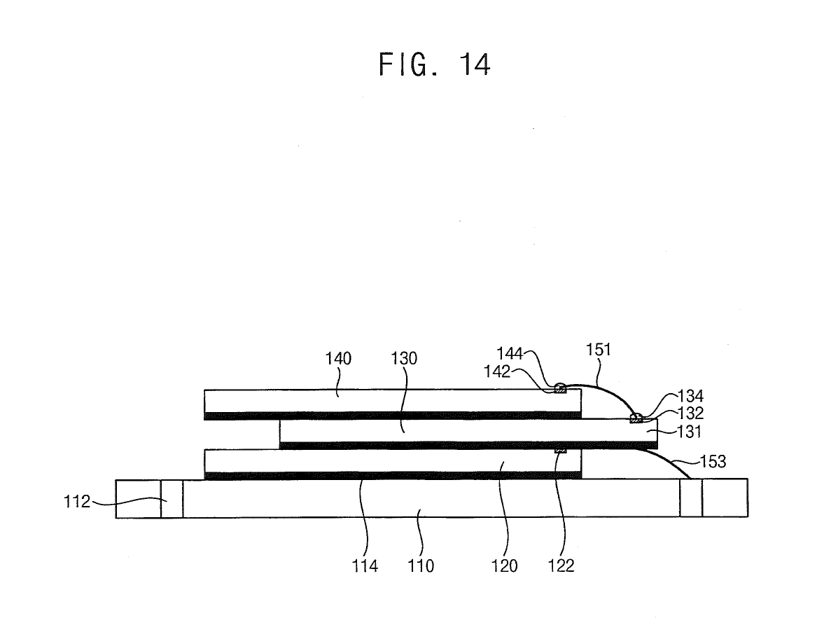

Referring now to FIG. 14, the metal line drawn through the capillary may be extended from the third pad bump 134 to the first pad bump 144 to form the first conductive wire 151 for electrically connecting the first pad bump 144 with the third pad bump 134.

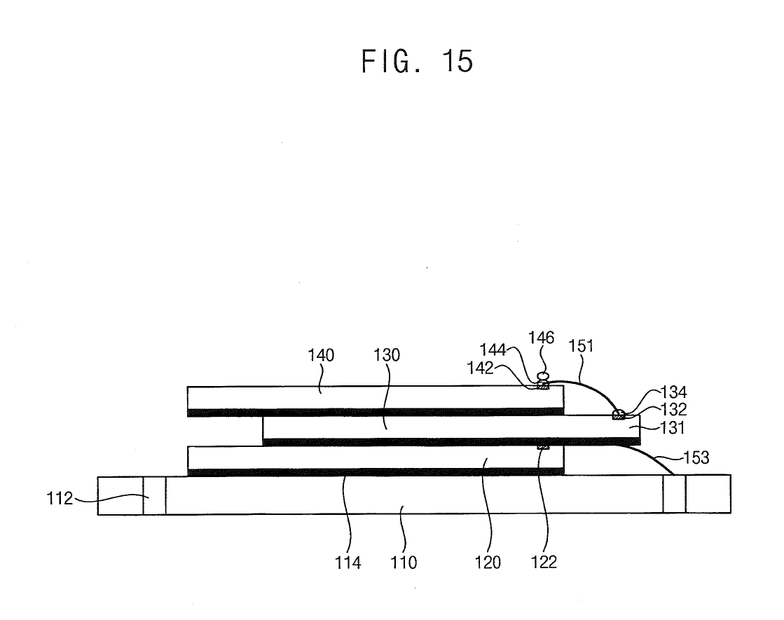

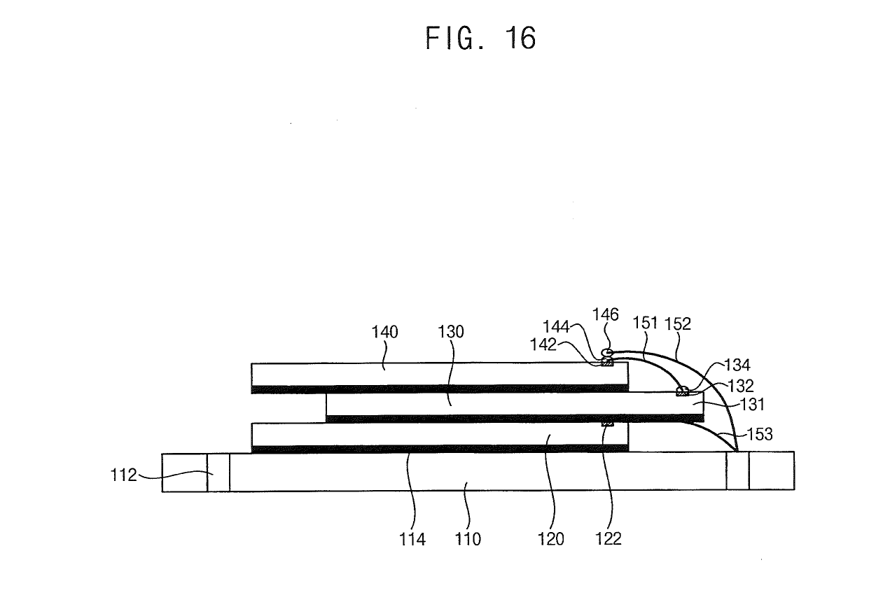

As illustrated in FIG. 15, the second pad bump 146 may be formed on the first pad bump 144. Referring to FIG. 16, the second pad bump 146 and the circuit pattern 112 of the package substrate 110 may be electrically connected with each other using the second conductive wire 152.

The control chip, the molding member 162 and the external terminals 164 may be sequentially formed to complete embodiments of the multi-chip package 100a illustrated in FIG. 10.

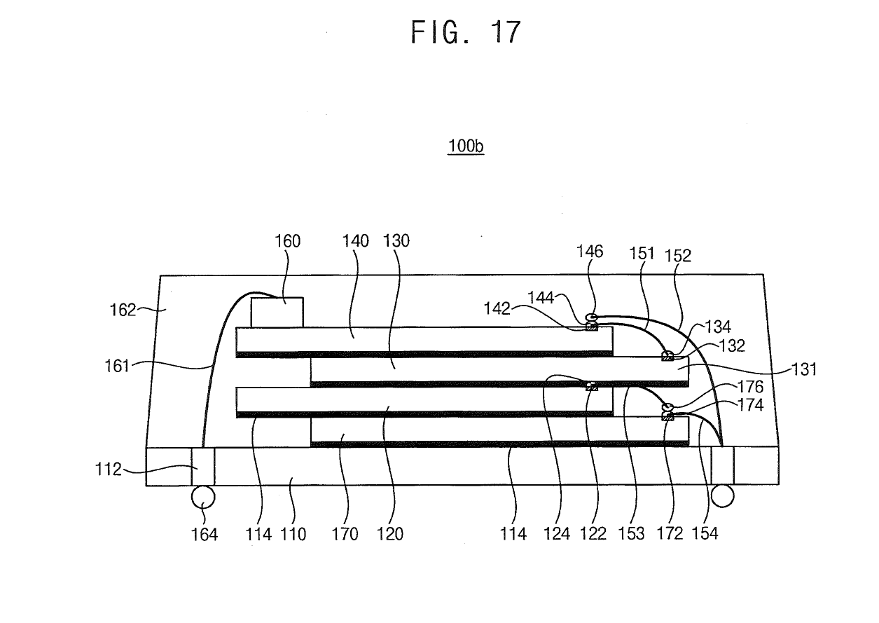

Referring now to FIG. 17, an enlarged cross-section illustrating a multi-chip package in accordance with some embodiments of the present inventive concept will be discussed. A multi-chip package 100b in these embodiments may include elements substantially the same as those of the multi-chip package 100a in FIG. 10, except embodiments illustrated in FIG. 17 include a fourth semiconductor chip. It will be understood that the same reference numerals refer to the same elements throughout and, therefore, details of elements already discussed with respect to previous embodiments will not be discussed again in the interest of brevity.

Referring now to FIG. 17, the multi-chip package 100b in these embodiments may further include the fourth semiconductor chip 170. The fourth semiconductor chip 170 may be between the package substrate 110 and the first semiconductor chip 120. In other words, the fourth semiconductor chip 170 may be positioned on a surface of the package substrate 110. The first semiconductor chip 120 may be arranged on a surface of the fourth semiconductor chip 170.

In some embodiments, the fourth semiconductor chip 170 may have a fourth bonding pad 172. The fourth bonding pad 172 may be arranged on an edge portion of the upper surface of the fourth semiconductor chip 170. The first semiconductor chip 120 may be positioned on the upper surface of the fourth semiconductor chip 170 to expose the fourth bonding pad 172.

In some embodiments, a fourth pad bump 124 may be formed on the first bonding pad 122. A fifth pad bump 174 may be formed on the fourth bonding pad 172. A sixth pad bump 176 may be formed on the fifth pad bump 174.

The third conductive wire 152 may be electrically connected between the fourth pad bump 124 and the sixth pad bump 176. A fourth conductive wire 154 may be electrically connected between the fifth pad bump 154 and the circuit pattern 112 of the package substrate 110. Thus, the first semiconductor chip 120 may be electrically connected with the package substrate 110 via the fourth semiconductor chip 170.

Alternatively, the multi-chip package 100b may not include the sixth pad bump 176. In these embodiments, the lower end of the third conductive wire 152 may be connected to the fifth pad bump 174.

In some embodiments, a stack structure of the fourth and first semiconductor chips 170 and 120 may be substantially the same as that of the second and third semiconductor chips 130 and 140. However, although the fourth semiconductor chip 170 may have a portion that protrudes from a side surface of the first semiconductor chip 120, the protruded portion of the fourth semiconductor chip 170 may make contact with a surface of the package substrate 110, so that the protruded portion of the fourth semiconductor chip 170 may be firmly supported by the package substrate 110. In other words, the fourth semiconductor chip 170 may not have an overhang, different from the second semiconductor chip 130. Therefore, although at least two wire bonding processes may be performed on the fourth bonding pad 172 on the protruded portion of the fourth semiconductor chip 170, cracks may not be generated in the fourth semiconductor chip 170.

Alternatively, the fourth conductive wire 154 may be electrically connected between the fourth pad bump 124 and the circuit pattern 112 of the package substrate 110. In these embodiments, the fourth semiconductor chip 170 may be electrically connected with the package substrate 110 via the first semiconductor chip 120.

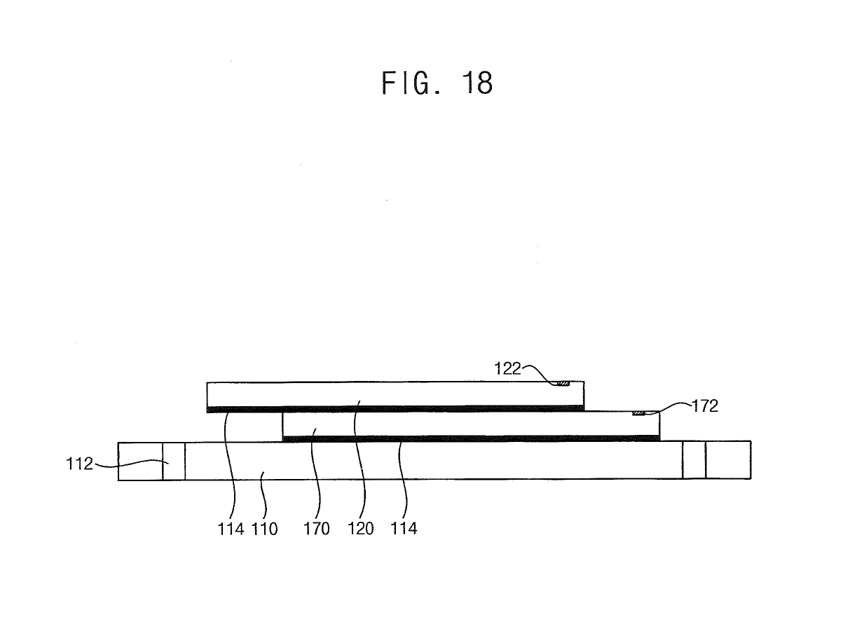

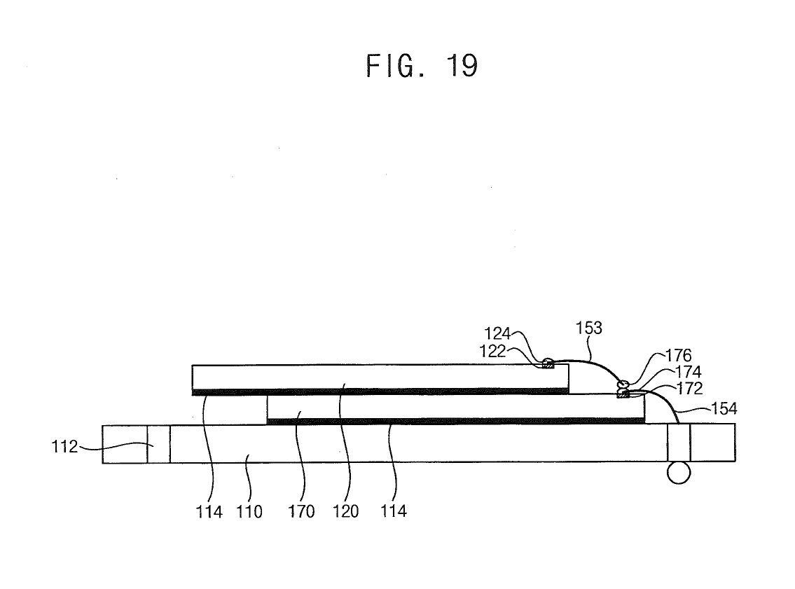

Referring now to FIGS. 18 and 19, cross-sections illustrating processing steps in the fabrication of multi-chip packages illustrated in FIG. 17 will be discussed. As illustrated in FIG. 18, the fourth semiconductor chip 170 may be attached to the upper surface of the package substrate 110 using the adhesive 114. The first semiconductor chip 120 may be attached to the upper surface of the fourth semiconductor chip 170 to expose the fourth bonding pad 172.

As illustrated in FIG. 19, the fourth pad bump 124 may be formed on the first bonding pad 122. The fifth pad bump 174 may be formed on the fourth bonding pad 172. The fifth pad bump 174 and the circuit pattern 112 of the package substrate 110 may be electrically connected with each other using the fourth conductive wire 154. The sixth pad bump 176 may be formed on the fifth pad bump 174. The fourth pad bump 124 and the sixth pad bump 176 may be electrically connected with each other using the third conductive wire 153.

Processes substantially the same as those illustrated with reference to FIGS. 12 to 16 may be performed to complete the multi-chip package 100b of FIG. 17.

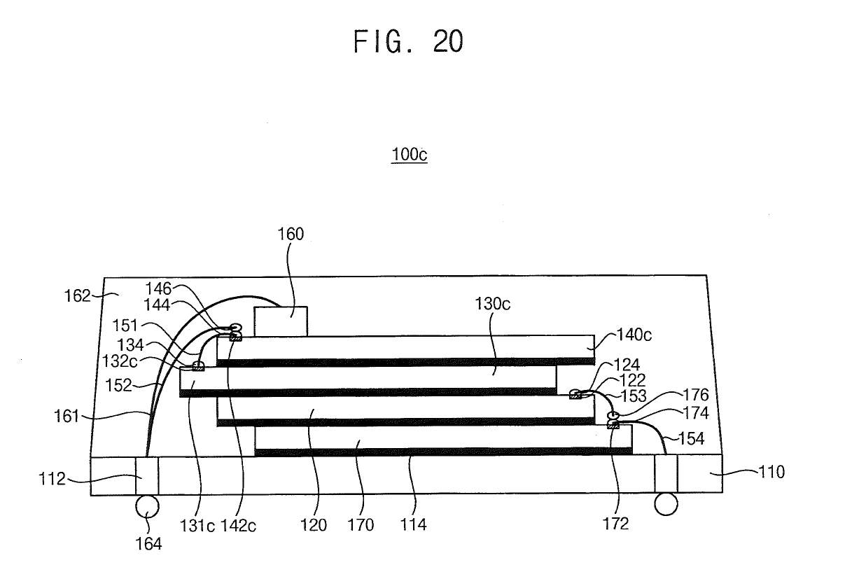

Referring now to FIG. 20, a cross-section illustrating a multi-chip package in accordance with some embodiments of the present inventive concept will be discussed. A multi-chip package 100c may include elements substantially the same as those of the multi-chip package 100b FIG. 17 except for positions of bonding pads and stack structures of semiconductor chips. It will be understood that the same reference numerals refer to the same elements throughout and, therefore, details of elements already discussed with respect to previous embodiments will not be discussed again in the interest of brevity.

Referring to now to FIG. 20, the second semiconductor chip 130c may have an overhang 131 c that horizontally protrudes from a left side surface of the first semiconductor chip 120. The third semiconductor chip 140c may be arranged on a surface of the second semiconductor chip 130c to expose the overhang 131c.

Thus, a second bonding pad 132c may be arranged on the overhang 131c corresponding to a left edge portion of the upper surface of the second semiconductor chip 130c. A third bonding pad 142c may be arranged on a left edge portion of an upper surface of the third semiconductor chip 140c.

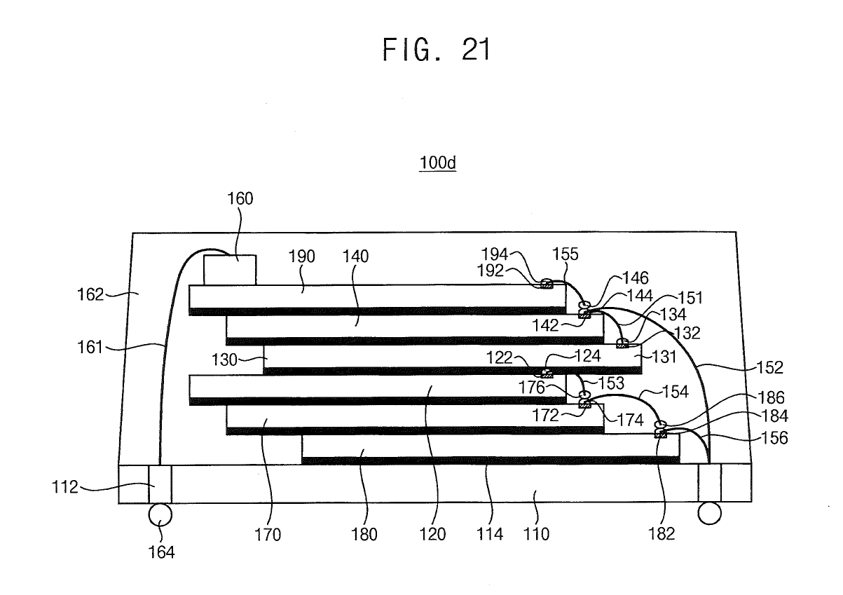

Referring now to FIG. 21, a cross-section illustrating a multi-chip package in accordance with some embodiments of the present inventive concept will be discussed.

A multi-chip package 100d may include elements substantially the same as those of the multi-chip package 100b discussed above with respect to FIG. 17 except for further including a fifth semiconductor chip and a sixth semiconductor chip. It will be understood that the same reference numerals refer to the same elements throughout and, therefore, details of elements already discussed with respect to previous embodiments will not be discussed again in the interest of brevity.

As illustrated in FIG. 21, the multi-chip package 100d may further include the fifth semiconductor chip 180 and a sixth semiconductor chip 190. The fifth semiconductor chip 180 may be between the package substrate 110 and the fourth semiconductor chip 170. Thus, the fifth semiconductor chip 180 may be arranged on a surface of the package substrate 110. The fourth semiconductor chip 170 may be arranged on a surface of the fifth semiconductor chip 180.

In some embodiments, the fifth semiconductor chip 180 may have a fifth bonding pad 182. The fifth bonding pad 182 may be arranged on a right edge portion of the upper surface of the fifth semiconductor chip 180. The fourth semiconductor chip 170 may be positioned on the upper surface of the fifth semiconductor chip 180 to expose the fifth bonding pad 182.

In some embodiments, a seventh pad bump 184 may be formed on the fifth bonding pad 182. An eighth pad bump 186 may be formed on the seventh pad bump 184. The fourth conductive wire 154 may be electrically connected between the fifth pad bump 174 and the eighth pad bump 186. A sixth conductive wire 156 may be electrically connected between the seventh pad bump 184 and the circuit pattern 112 of the package substrate 110.

In some embodiments, the multi-chip package 100d may not include the eighth pad bump 186. In these embodiments, the lower end of the fourth conductive wire 154 may be connected to the seventh pad bump 184.

The sixth semiconductor chip 190 may be stacked on the third semiconductor chip 140. The sixth semiconductor chip 190 may have a sixth bonding pad 192. The sixth semiconductor chip 190 may be positioned on the surface of the third semiconductor chip 140 to expose the third bonding pad 142.

A fifth conductive wire 155 may be electrically connected between the second bonding pad 142 and the sixth bonding pad 192. In some embodiments, a ninth pad bump 194 may be formed on the sixth bonding pad 192. A first end of the fifth conductive wire 155 may be connected to the ninth pad bump 194. A second end of the fifth conductive wire 155 may be connected to the second pad bump 146. Thus, the sixth semiconductor chip 190 may be electrically connected with the package substrate 110 via the third semiconductor chip 140.

Processing steps in the manufacturing of multi-chip package 100d may include processes substantially the same as those for manufacturing the multi-chip package 100b discussed with respect to FIG. 17 except for further including wire bonding processes on the fifths semiconductor chip 180 and the sixth semiconductor chip 190. Thus, the details of the processing steps for manufacturing the multi-chip package 100d may be omitted herein for brevity.

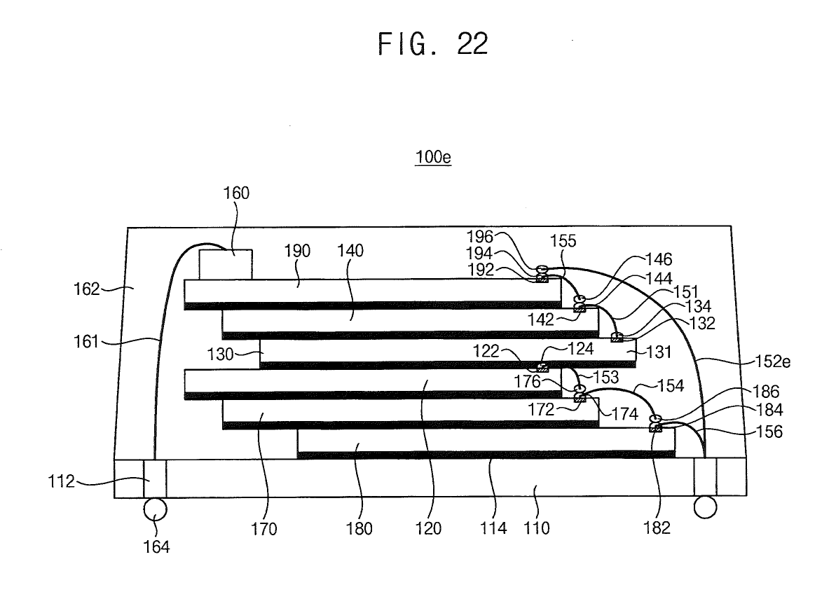

Referring now to FIG. 22, a cross-section illustrating a multi-chip package in accordance with some embodiments of the present inventive concept will be discussed. A multi-chip package 100e may include elements substantially the same as those of the multi-chip package 100d discussed above with respect to FIG. 21 except for a second conductive wire. It will be understood that the same reference numerals refer to the same elements throughout and, therefore, details of elements already discussed with respect to previous embodiments will not be discussed again in the interest of brevity.

As illustrated in FIG. 22, the second conductive wire 152e of the multi-chip package 100d may be electrically connected between the sixth bonding pad 192 of the sixth semiconductor chip 190 and the circuit pattern 112 of the package substrate 110.

In some embodiments, a tenth pad bump 196 may be formed on the ninth pad bump 194. A first end of the second conductive wire 152e may be connected to the tenth pad bump 196. Thus, the third semiconductor chip 140 may be electrically connected with the package substrate 110 via the sixth semiconductor chip 190. The sixth semiconductor chip 190 may be directly connected with the package substrate 110 via the second conductive wire 152e.

Processing steps in the manufacturing of multi-chip packages 100e may include processes substantially the same as those for manufacturing the multi-chip package 100d discussed above with respect to FIG. 21 except for a process for connecting the upper end of the second conductive wire 152e to the tenth pad bump 196. Thus, details of the processing steps in the manufacturing of the multi-chip package 100e may be omitted herein for brevity.

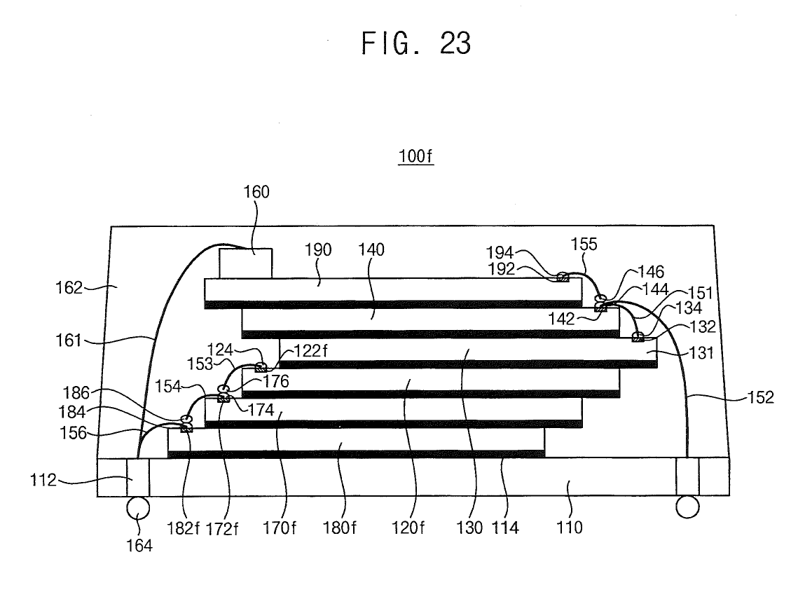

Referring now to FIG. 23, a cross-section illustrating a multi-chip package in accordance with some embodiments will be discussed. A multi-chip package 100f may include elements substantially the same as those of the multi-chip package 100d discussed above with respect to FIG. 21 except for positions of bonding pads and stack structures of semiconductor chips. It will be understood that the same reference numerals refer to the same elements throughout and, therefore, details of elements already discussed with respect to previous embodiments will not be discussed again in the interest of brevity.

As illustrated in FIG. 23, a first bonding pad 122f may be arranged on a left edge portion of a surface of a first semiconductor chip 120f. A fourth bonding pad 172f may be arranged on a left edge portion of a surface of a fourth semiconductor chip 170f. A fifth bonding pad 182f may be arranged on a left edge portion of a surface of a fifth semiconductor chip 180f.

In some embodiments, the second semiconductor chip 120 may be arranged on the a surface of the first semiconductor chip 120f to expose the first bonding pad 122f. The first semiconductor chip 120f may be arranged on the surface of the fourth semiconductor chip 170f to expose the fourth bonding pad 172f. The fourth semiconductor chip 170f may be arranged on the surface of the fifth semiconductor chip 180f to expose the fifth bonding pad 182f.

Processing steps in the manufacturing of the multi-chip package 100f may include processes substantially the same as those for manufacturing the multi-chip package 100d discussed above with respect to FIG. 21. Thus, details of the processing steps of manufacturing the multi-chip package 100f may be omitted herein for brevity.

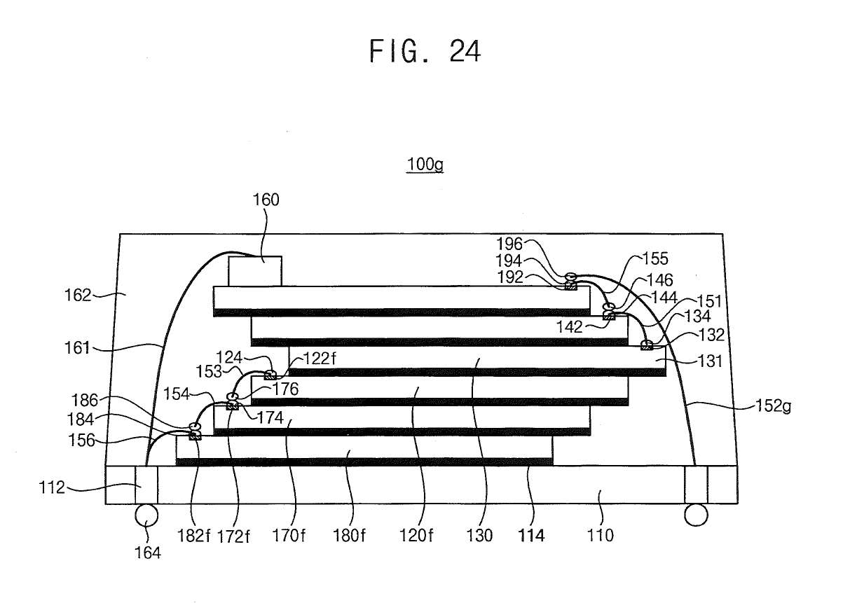

Referring now to FIG. 24, a cross-section illustrating a multi-chip package in accordance with some embodiments of the present inventive concept will be discussed. A multi-chip package 100g may include elements substantially the same as those of the multi-chip package 100f discussed above with respect to FIG. 23 except for a second conductive wire. It will be understood that the same reference numerals refer to the same elements throughout and, therefore, details of elements already discussed with respect to previous embodiments will not be discussed again in the interest of brevity.

Referring now to FIG. 24, the second conductive wire 152g of the multi-chip package 100g may be electrically connected between the sixth bonding pad 192 of the sixth semiconductor chip 190 and the circuit pattern 112 of the package substrate 110.

In some embodiments, a tenth pad bump 196 may be formed on the ninth pad bump 194. A first end of the second conductive wire 152e may be connected to the tenth pad bump 196. Thus, the third semiconductor chip 140 may be electrically connected with the package substrate 110 via the sixth semiconductor chip 190. The sixth semiconductor chip 190 may be directly connected with the package substrate 110 via the second conductive wire 152g.

Processing steps in the manufacturing of the multi-chip package 100g may include processes substantially the same as those for manufacturing the multi-chip package 100f discussed above with respect to FIG. 23 except for a process for connecting the end of the second conductive wire 152g to the tenth pad bump 196. Thus, details of the processing steps in the manufacturing of the multi-chip package 100g may be omitted herein for brevity.

According to some embodiments discussed herein, only one conductive wire may be connected to the bonding pad on the overhang. In other words, only one wire bonding process may be performed on the weak overhang. Thus, stresses applied to the overhang may be remarkably reduced. As a result, generations of cracks in the semiconductor chip having the overhand may be suppressed.

Further, because it may not be required to provide the semiconductor chip having the overhang with strength stronger than that of other semiconductor chips without the overhang, the semiconductor chip having the overhang may have a thickness of no more than thickness of other semiconductor chips without the overhang. Therefore, the multi-chip package including the semiconductor chip having the overhang may have a relatively thin thickness.

The foregoing is illustrative of example embodiments and is not to be construed as limiting thereof. Although a few example embodiments have been described, those skilled in the art will readily appreciate that many modifications are possible in the example embodiments without materially departing from the novel teachings and advantages of the present inventive concept. Accordingly, all such modifications are intended to be included within the scope of the present inventive concept as defined in the claims. In the claims, means-plus-function clauses are intended to cover the structures described herein as performing the recited function and not only structural equivalents but also equivalent structures. Therefore, it is to be understood that the foregoing is illustrative of various example embodiments and is not to be construed as limited to the specific example embodiments disclosed, and that modifications to the disclosed example embodiments, as well as other example embodiments, are intended to be included within the scope of the appended claims.

What is claimed is:

1. A multi-chip package comprising:
a package substrate;
a first semiconductor chip on a first surface of the package substrate, the first semiconductor chip having a first bonding pad that is electrically coupled to the package substrate;
a second semiconductor chip arranged on a first surface of the first semiconductor chip, the second semiconductor chip having an overhang that protrudes from a side surface of the first semiconductor chip and a second bonding pad on the overhang;
a third semiconductor chip arranged on a first surface of the second semiconductor chip to expose the overhang, the third semiconductor chip having a third bonding pad;
a first conductive wire that electrically connects the second bonding pad and the third bonding pad; and
a second conductive wire that electrically connects the third bonding pad and the package substrate.

2. The multi-chip package of claim 1, further comprising:
a fourth semiconductor chip between the package substrate and the first semiconductor chip, the fourth semiconductor chip having a fourth bonding pad;
a third conductive wire that electrically connects the first bonding pad and the fourth bonding pad; and
a fourth conductive wire that electrically connects the fourth bonding pad and the package substrate.

3. The multi-chip package of claim 1, further comprising:
a fifth semiconductor chip on a first surface of the third semiconductor chip, the fifth semiconductor chip having a fifth bonding pad; and
a fifth conductive wire that electrically connects the fifth bonding pad and the third bonding pad.

4. The multi-chip package of claim 1, further comprising:
a fifth semiconductor chip on a first surface of the third semiconductor chip, the fifth semiconductor chip having a fifth bonding pad that is connected to the second conductive wire; and
a fifth conductive wire that electrically connects the fifth bonding pad and the package substrate.

5. The multi-chip package of claim 1:
wherein the first bonding pad is positioned on a first edge portion of the first surface of the first semiconductor chip adjacent to the overhang; and
wherein the second semiconductor chip is positioned on the first surface of the first semiconductor chip to cover the first bonding pad.

6. The multi-chip package of claim 1:
wherein the first bonding pad is positioned on a second edge portion of the first surface of the first semiconductor chip opposite the overhang; and
wherein the second semiconductor chip is positioned on the first surface of the first semiconductor chip to expose the first bonding pad.

7. The multi-chip package of claim 1, further comprising a first pad bump on the third bonding pad, the first pad bump being connected to an end of the first conductive wire.

8. The multi-chip package of claim 7, further comprising a second pad bump on the first pad bump, the second pad bump being connected to the second conductive wire.

9. The multi-chip package of claim 1, further comprising a third pad bump on the second bonding pad, the third pad bump being connected to an end of the first conductive wire.

10. The multi-chip package of claim 1, wherein a thickness of the second semiconductor chip is no greater than a thickness of the first semiconductor chip or the third semiconductor chip.

11. The multi-chip package of claim 1, further comprising:
a molding member on the first surface of the package substrate that covers the first, second and third semiconductor chips; and
external terminals on a second surface, opposite the first surface, of the package substrate.

12. A method of manufacturing a multi-chip package, the method comprising:
arranging a first semiconductor chip on a first surface of the package substrate, the first semiconductor chip having a first bonding pad;
arranging a second semiconductor chip on a first surface of the first semiconductor chip, the second semiconductor chip having an overhang that protrudes from a side surface of the first semiconductor chip and a second bonding pad arranged on the overhang;
arranging a third semiconductor chip on a first surface of the second semiconductor chip to expose the overhang, the third semiconductor chip having a third bonding pad;
electrically connecting the second bonding pad to the third bonding pad using a first conductive wire; and
electrically connecting the third bonding pad to the package substrate using a second conductive wire.

13. The method of claim 12, further comprising:
a fourth semiconductor chip between the package substrate and the first semiconductor chip, the fourth semiconductor chip having a fourth bonding pad;
electrically connecting the first bonding pad to the fourth bonding pad using a third conductive wire; and
electrically connecting the fourth bonding pad to the package substrate using a fourth conductive wire.

14. The method of claim 12, further comprising:
arranging a fifth semiconductor chip on a first surface of the third semiconductor chip, the fifth semiconductor chip having a fifth bonding pad; and
electrically connecting the fifth bonding pad to the third bonding pad using a fifth conductive wire.

15. The method of claim 12, further comprising:
arranging a fifth semiconductor chip on a first surface of the third semiconductor chip, the fifth semiconductor chip having a fifth bonding pad;
electrically connecting the second conductive wire to the fifth bonding pad; and
electrically connecting the fifth bonding pad to the package substrate using a fifth conductive wire.
